(19) United States
(12) Patent Application Publication
(10) Pub. No.: US 2026/0335272 A1
(43) Pub. Date: Oct. 29, 2026

(54) SAGERINIC ACID AND COMPOSITIONS CONTAINING SAME, FOR USE IN THE PREVENTION AND/OR TREATMENT OF SENESCENCE-RELATED DISORDERS IN A HUMAN OR ANIMAL

(71) Applicant: A2P SCIENCES, Lyon (FR)

(72) Inventors: Daniel JEAN, Vic-le-Comte (FR); Louis ASTIER, Clermont-Ferrand (FR); Audrey PICAUD, Clermont-Ferrand (FR); Antoine PELLET, Lyon (FR)

(21) Appl. No.: 19/114,521

(22) PCT Filed: Sep. 23, 2022
(86) PCT No.: PCT/FR2022/051796

§ 371 Date: Mar. 24, 2025

(51) Int. Cl. A61K 31/216; A61K 9/06; A61K 9/48; A61K 31/05; A61K 31/426; A61K 36/53; A61P 39/06

(52) U.S. Cl. CPC A61K 31/216; A61K 9/06; A61K 9/48; A61K 31/05; A61K 31/426; A61K 36/53; A61P 39/06

(57) ABSTRACT

The disclosure concerns the sagerinic acid and the compositions containing it for use in the prevention and/or the treatment of the disorders related to senescence in humans and animals.

The invention relates to sagerinic acid and the compositions containing it, for use in the prevention and/or treatment of disorders related to senescence in humans and animals.

Ageing is a multifactorial process characterized by the progressive loss of physiological functions, leading to increased vulnerability to age-related diseases. Several theories have been proposed to explain the nature of ageing and there is now a consensus on how to categorize the different mechanisms involved in the ageing.

One of the best known identifies the free radicals produced by the mitochondrial metabolism as the cause of the cellular disorders and damage to genetic material (1).

Another well-known aspect of ageing is the alteration of the genetic material over time. There is considerable evidence that the epigenetic modifications, such as DNA methylation, non-coding RNA and histone modifications, play an essential role in the molecular mechanism of the ageing. The calorie restriction is the intervention which, in all the animal species to which it has been applied, has produced beneficial effects on the health of ageing organisms and significantly increased their longevity. It has been shown that the sirtuins, which are histone diacylation enzymes that protect the genetic material from damage induced by various factors, are strongly positively involved in the effects of the calorie restriction (2).

Another aspect of ageing is protein glycation (3). Generally speaking, all proteins interact with reducing oses in a passive, spontaneous reaction that does not involve an enzyme, resulting in condensed compounds that are more or less irreversible. This reaction is known as the Maillard reaction, the mechanisms of which were first described by J. E. Hodge in 1953 (4). This reaction gives rise to multiple rearranged compounds (Amadori bodies, among others). When this reaction is completed, we speak of “Advanced Glycation Endproducts” or AGEs.

This reaction is universal. It occurs in all cases, but with varying degrees of intensity depending on the sugars involved and the number of free amino radicals present on the protein molecule. This reaction is amplified by heat and depends on the concentration of sugars present in the medium.

During glycation, the proteins undergo “crosslinks” that alter their functionality and stiffen the structural proteins such as collagen and elastin. These proteins are normally eliminated by proteases which allow them to be renewed. The more advanced the glycation, the less sensitive the proteins are to the effect of proteases. The slow-renewing proteins, such as those found in joints or the brain, undergo particularly a strong glycation linked to this slow rate of renewal.

The ageing therefore leads passively to an increased glycation of proteins with low renewal. Diabetes leads to the same result because of high blood sugar levels. In both cases, there is a weakening of the organs whose functions depend on the connective tissue, particularly the vascular system.

As a result, the diabetic disease is generally associated with various angiopathies, particularly in the eyes and kidneys.

The disorders associated with glycation, whether normal, i.e. correlated with physiological ageing, or pathological as in diabetes (5), are very varied and of varying severity. One example is the loss of elasticity in blood and lymphatic vessels, which leads to all forms of angiopathy associated with diseases such as stroke, atheroma, macular degeneration, renal failure (6) and respiratory failure. Aesthetic damage to the integumentary system, such as sagging skin, is also a problem.

Generally speaking, it also seems to be accepted that the glycation of the collagen, the most important protein in mammals, is a reliable marker of biological age and can be used to determine life expectancy (7, 8).

Ageing leads to remodeling of the large arteries. These age-related changes may be associated with an increased activity of the metalloproteinases, particularly the collagenases and the elastases in large-diameter arteries (9). It is therefore important that the activity of these enzymes is perfectly regulated. This regulation can be achieved by protecting the proteins of the arterial connective tissue, mainly collagen and elastin, against the excessive action of these enzymes.

As a result of the above, to act effectively against the ageing of the body, it would be desirable to act simultaneously on what appear to be the four fundamental axes of ageing:
the oxidative stress through the use of anti-radical molecules
the glycation of the proteins by molecules capable of reversing glycation that has already taken place
the DNA protection through activation of sirtuins
the protection of the connective tissue by inhibiting the proteases that break it down, in particular the elastases and collagenases.

Molecules capable of acting on one or more of these four axes have already been described.

### The Anti-Oxidants and Free Radical Traps

Numerous antioxidants and free radical traps have been described since the beginning of the twentieth century, particularly in the plant kingdom, where the polyphenols are the most common representatives. There are also molecules such as water-soluble vitamin C and fat-soluble vitamin E, which have a good anti-radical properties. A simple and rapid method for assessing the anti-radical effect implements a stable free radical, 22-diphenyl 1-pycrylhydrazyl (DPPH), whose degradation by the molecule to be tested is measured colorimetrically.

### The Glycation Inhibitors and the Protein Deglycants

A large number of molecules are currently known to inhibit protein glycation (10). Most simply act as antioxidants, since the glycation reaction comprises an oxidative phase. So, for example, a large number of polyphenols have been recognized as being able to be used as inhibitors of the protein glycation reaction. Others, such as aminoguanidine, carnosine (a monopeptide found in red meat) and pyridoxamine, act as competitive inhibitors of the amino radicals regarding the amino radicals of the oses involved in glycation.

The glycation inhibitors of the proteins are of benefit to health by limiting this reaction and reducing the production of new cross-links. However, the glycation slows down the renewal of the proteins, leading to their accumulation and the functional degradation of the organs to which they are linked. Some proteins renew themselves slowly, in particular the proteins of the connective tissue, the collagen and the elastin, which play a key role in the viscoelastic properties of the cardiovascular system, joints and dermis. It is therefore extremely important to discover molecules capable of reversing glycation, so as to cut the pre-existing cross-links and allow the tissues to regain at least some of their original viscoelastic properties.

In particular, the reduction in the elasticity of the cardiovascular system is the most important problem during ageing and the main cause of this reduction is the formation of AGE and cross-links between protein molecules.

The use of a glycation inhibitor does not restore to this system the elasticity lost over time. This requires an agent capable of cutting cross-links.

There is therefore a clear need for products capable of breaking down the protein cross-links created by the glycation, without any harmful side-effects.

The applications for such products are considerable. Among the most important are the fight against the angiopathies and in particular the associated nephropathies, cardiopathies and retinopathies, the improvement of blood and lymphatic vascular function in the elderly, the improvement of the skin elasticity leading to an improved appearance, and the fight against the loss of elasticity in tendons and cartilage, leading to reduced mobility due to inflammation and pain in the joints. All these pathologies are associated with age in normal subjects and are aggravated by diabetes. Another example is the improvement in venous and lymphatic function when resolving trauma by improving the functionality of connective tissue proteins thanks to a reduction in their glycation rate.

Finally, the ageing is accompanied by a degradation of the vascular connective tissue, leading to the stiffening of the blood and lymphatic vessels and the creation of atheroma plaques.

Certain molecules in the thiazolium group have been described in U.S. Pat. No. 5,853,703A (22, 23) as having this activity in vitro and in animal pharmacology studies. The patent WO2011042890A2 reveals a family of polyphenolic molecules with a protein deglycation effect (11).

### The Sirtuin Activators (12)

The lysine deacylase proteins dependent of the NAD+ of the sirtuin family regulate a range of physiological functions, from energy metabolism to stress responses. The human isoforms of Sirtuin, SIRT1-7, which are particularly activated during calorie restriction, are considered to be attractive therapeutic targets for the age-related diseases such as type 2 diabetes, inflammatory and neurodegenerative diseases. Pharmacological activators of sirtuin-1 and other isoforms have been described, and initial clinical trials have been performed.

### The Protectors of the Connective Tissue (13)

Epidemiological studies have suggested an association between the consumption of foods or drinks containing polyphenols and the prevention of certain human diseases, such as chronic obstructive pulmonary and cardiac disease, chronic inflammation, as well as reducing the risk of many types of cancer. It now seems proven that polyphenols protect the proteins of the extracellular matrix against the attacks from proteases over-expressed during exposure to free radicals and inflammation.

However, none of these molecules alone can act simultaneously on the four fundamental aspects of ageing:
the oxidative stress through the use of anti-radical molecules
the glycation of the proteins by molecules capable of reversing glycation that has already taken place
the DNA protection through activation of sirtuins
the protection of the connective tissue by inhibiting the proteases that break it down, in particular the elastases and collagenases.

Sagerinic acid is a molecule that was discovered and described for the first time in common sage (Salvia officinalis) by Yinrong Lu and L. Yeap Foo in 1999 (17).

Sagerinic acid has since been identified in a large number of plant species in the Lamiaceae family, including Melissa officinalis (18), Rosmarinus officinalis (19), Orthosiphon grandiflorus (20), Helicteres hirsuta (21) and Plectranthus amboinicus (15).

Sage and lemon balm are plants with multiple applications and properties, and these multiple effects are generally due to the presence of different molecules in the extracts.

The efficacy of sagerinic acid as an inhibitor of COVID19 was predicted by in silico simulation, but no physical evidence of this activity has been produced (14).

In addition, the U.S. Pat. No. 8,105,636 B2 (15) claims a composition comprising an extract of Plectranthus amboinicus as an anti-inflammatory wherein sagerinic acid is cited as one of the possible active ingredients.

The potential antileishmanial and anti-inflammatory activity of sagerinic acid has been described (16).

The invention is based on the fortuitous discovery of the effects of sagerinic acid on these four fundamental aspects of ageing: during in vitro tests, it was observed that, surprisingly, a single molecule, sagerinic acid, possesses the four properties described above.

The invention applies to the prevention and to the treatment of the disorders related to senescence.

These include, but are not limited to, the following:
connective tissue diseases affecting cartilage, such as articular osteoarthritis and rheumatoid arthritis in their non-inflammatory components (degradation of the connective tissue, not immune reactions leading to the inflammation),
the diseases of the vascular connective tissue: strokes, vascular nephritis and retinitis, macular degeneration, atheromatous disease,
the diseases directly linked to protein glycation, such as cataracts, Alzheimer's disease, skin healing disorders and presbycusis,
the aesthetic skin disorders such as lipofuscin accumulation, pigmentation spots, erythema and loss of skin elasticity.

The sagerinic acid can be applied in pure form or as an extract, combined with excipients suited to its use: oral, parenteral, topical, rectal or inhalation.

The invention therefore proposes the sagerinic acid for use in the prevention and/or the treatment of the disorders related to senescence in a human or animal.

In a first embodiment, these disorders are due to oxidative stress.

In a second embodiment, these disorders are due to protein glycation.

In a third embodiment, these disorders are due to an alteration in the DNA.

In a fourth embodiment, these disorders are due to an alteration in the proteins of the arterial connective tissue.

The invention also offers the sagerinic acid for a use as an antioxidant agent and/or free radical trap.

The invention also offers the sagerinic acid for a use as a protein deglycation agent.

The invention also offers sagerinic acid for a use as a DNA protection agent.

The invention also proposes sagerinic acid for a use as a protease inhibition agent.

In a preferred embodiment, the invention proposes the sagerinic acid for use in the prevention and/or the treatment, simultaneously, of disorders due to oxidative stress, and disorders due to protein glycation, and disorders due to an alteration in the DNA and disorders due to an alteration in the proteins of the arterial connective tissue, in a human or animal.

Another object of the invention is a composition comprising sagerinic acid for use in the prevention and/or the treatment of disorders related to senescence in a human or animal.

In a first embodiment of the composition of the invention, the disorders are due to an oxidative stress.

In a second embodiment of the composition of the invention, the disorders are due to protein glycation.

In a third embodiment of the composition of the invention, the disorders are due to an alteration in the DNA.

In a fourth embodiment of the composition of the invention, the disorders are due to an alteration in the proteins of the arterial connective tissue.

A preferred object of the invention is a composition comprising sagerinic acid for use in the simultaneous prevention and/or treatment of disorders due to an oxidative stress, and disorders due to protein glycation, and disorders due to an alteration in the DNA and disorders due to an alteration in the proteins of the arterial connective tissue, in a human or animal.

The invention will be better understood and its characteristics and advantages will become clearer on reading the explanatory description and the examples of use that follow.

### EXAMPLES

### Example 1: Preparation of Purified Sagerinic Acid

170 g of dried lemon balm (Melissa officinalis) leaves are macerated in 2 L of deionized water at room temperature under constant stirring for 24 hours.

The extractive solution is separated by filtration on paper and then lyophilized. The weight of the extraction residue was 32.3 g, giving a yield of 19%.

10 g of lyophilized extract is dissolved in 50 mL of deionized water and this solution is chromatographed on a column 45 mm in diameter and 24 cm long containing approximately 200 g of Daïaon HP20 resin (Thermo Scientific ref 046488.A1) with 2 L of deionized water as eluent.

The 2 L eluted from the column are eliminated. It is then eluted with 2 L of methanol (Thermo Scientific ref. L13255). All the eluate is recovered and evaporated under reduced pressure until the methanol has evaporated completely. The remaining 100 mL aqueous phase is made up with deionized water, and 10 mL of concentrated hydrochloric acid (Honeywell Fluka, ref. 320331) and are countercurrent extracted by 3 times 100 ml of ethyl acetate (Roth Ref. 73361).

The organic phase is dried over anhydrous sodium sulphate (Sigma Aldrich, ref 238597), filtered and evaporated to dryness. Approximately 700 mg of partially purified sagerinic acid was obtained.

The 700 mg of crude sagerinic acid was then chromatographed in batches of 100 mg dissolved in 1 mL of deionized water by preparative HPLC on a 22 mm diameter, 250 mm long C18 column (Vydac Denali 10 μm) using the following gradient:

Flow rate: 20 mL/min

Solvent A: Deionized water containing 0.1% formic acid (Fischer Chemical A117-50)

Solvent B: methanol (Thermo Scientific ref L13255)

| Time (min) | % Solvent A | % solvent B |
|---|---|---|
| 0 | 100 | 0 |
| 30 | 60 | 40 |
| 35 | 0 | 100 |
| 40 | 0 | 100 |
| 45 | 100 | 0 |

Fractions of 10 ml are collected.

The fraction containing sagerinic acid is identified by its UV spectrum (max). It is kept and the other fractions are eliminated.

After passing the 700 mg, the fractions are combined and evaporated to dryness under reduced pressure. A residue of 165 mg was obtained.

The remaining 165 mg were then chromatographed in the same way as above. The final product is 23 mg of 92% pure sagerinic acid.

### Example 2: Preparation of Lemon Balm Extract Enriched with Sagerinic Acid

170 g of dried lemon balm leaves are macerated in 2 L of deionized water at room temperature under constant stirring for 24 hours.

The extractive solution is separated by filtration on paper and then lyophilized. The weight of the extraction residue was 32.3 g, giving a yield of 19%.

10 g of lyophilized extract was dissolved in 50 mL of deionized water and chromatographed on a column 45 mm in diameter and 24 cm long containing approximately 200 g of Daïaon HP20 resin (Thermo Scientific ref. 046488.A1) with 2 L of deionized water as eluent.

The 2 L eluted from the column are eliminated. It is then eluted with 2 L of methanol (Thermo Scientific ref. L13255). All the eluate is recovered and evaporated under reduced pressure until the methanol has evaporated completely. The remaining 100 mL aqueous phase is made up with deionized water, and 10 mL of concentrated hydrochloric acid (Honeywell Fluka, ref. 320331) and are countercurrent extracted by 3 times 100 ml of ethyl acetate (Roth Ref. 73361).

The organic phase is dried over anhydrous sodium sulphate (Sigma Aldrich, ref 238597), filtered and evaporated to dryness. Approximately 700 mg of lemon balm extract containing 8% sagerinic acid is obtained.

### Example 3: Evaluation of the Effects of Sagerinic Acid and Ascorbic Acid on Oxidative Stress

### 3.1. Principle

The principle of the method for assessing the effect of sagerinic acid compared to ascorbic acid as a reference molecule is based on the elimination of a stable free radical, the 2,2-Diphenyl-1-picrylhydrazyl (DPPH Sigma Aldrich ref D9132).

### 3.2. Apparatus and Materials

Jasco V-730 Dual Beam UV-Visible Spectrophotometer
Stopwatch
Standard laboratory equipment.
Precision scales: ML104 (Mettler Toledo).
Automatic variable volume pipettes.

### 3.3. Reagents

Monopotassium phosphate (Riedel de Haen, ref. 04243)
Dibasic sodium phosphate dihydrate (Sigma Aldrich ref 30435)
Purified water
DPPH (Sigma Aldrich, ref. D9132)
Methanol Optima® LC/MS Grade (Fisher Chemical, ref.A456-212)
Toluene, Reference (Carl Roth ref. 4445.1)
Pharmethyl 96° ethyl alcohol, (Cristalco ref 11371014)

### 3.4. Preparation of Reagent Solutions

### Phosphate Buffer pH 7

Dissolve 0.177 g monopotassium phosphate and 0.730 g dibasic sodium phosphate dihydrate in 50 ml purified water. The pH is adjusted if necessary.

Solution of 2,2-diphenyl-1-pycrilhydrazyl (DPPH) in Methanol

A solution of exactly 0.0027% (w/V) DPPH in methanol is made.

### Antioxidant Solution

A solution of exactly 0.1% (w/V) ascorbic acid (Riedel-de-Haen, ref.33034) is made in ethyl alcohol. This solution is diluted to 0.0007% (V/V) in ethyl alcohol.

A solution of exactly 0.1% (w/V) sagerinic acid (prepared according to example 5.1.) is made in water. This solution is diluted to 0.0007% (V/V) in water.

### 3.5. Operating Mode

### 3.5.1. Control Solution:

A series of hermetically sealed pillboxes are filled with:

| | Sample | Blank |
|---|---|---|
| Buffer (ml) | 1.50 | 1.50 |
| DPPH solution (ml) | 3.00 | 0.00 |
| Methanol (ml) | 0.00 | 3.00 |

### 3.5.2. Test:

A series of hermetically sealed pillboxes are filled with:

| | Sample | Blank |
|---|---|---|
| Buffer (ml) | 1.35 | 1.35 |
| DPPH solution (ml) | 3.00 | 0.00 |
| Methanol (ml) | 0.00 | 3.00 |
| Antioxidant solution to test | 0.15 | 0.15 |

For the sample and the blank:
After addition, stir for 5 min on a magnetic stirrer
Add 4 ml toluene
Stir for 30 seconds with a magnetic stirrer
Allow to settle for 20 seconds to 1 minute (if necessary)
Recover the organic phase from the top using an automatic pipette, taking care not to recover any aqueous phase
Read the OD of the organic phase using a spectrophotometer at 519 nm

### 3.6. Results

| Molecule tested | Activity (arbitrary dimensionless unit) |
|---|---|
| Ascorbic acid (reference) | 5.26 |
| Sagerinic acid | 9.70 |

These results show that sagerinic acid has almost twice the effect of ascorbic acid in eliminating free radicals and therefore in preventing and/or treating disorders caused by oxidative stress

### Example 4: Evaluation of the Effects of Sagerinic Acid and Alagebrium as Protein Deglycants

### 4.1. Principle

The test products in 0.25% (w/V) solution are left in contact with a 0.375% (w/V) solution of 1-phenyl-1,2-propanedione (PPD) for 24 hours at 37 °C. An HPLC dosing, at 229 nm after passage through an Ascentis® Express C18 column, was carried out at TO and again after 24 hours so as to determine the benzoic acid content. Calculating the percentage of pmol of benzoic acid/μmol of PPD in the reaction medium determines the activity of the deglycants.

### 4.2. Apparatus and Materials

Ultimate 3000 HPLC system (Thermo Scientific—Dionex) with diode array detector and column oven.
Ascentis® Express C18 HPLC column (Supelco Ref 53816-U)
   Dimensions: 15 cm×3.0 mm
   Particle size: 2.7 μm
BD 53/E2 Oven (Binder).
0.45 μm pore size PVDF (Polyvinyl Fluoride) filters (Millex®-HV Ref SLHVX13NL).
Standard laboratory equipment.
Precision scales: ML104 (Mettler Toledo).
Automatic variable volume pipettes.

### 4.3. Reagents

Water Optima® LC/MS Suitable for UHPLC-UV (Fisher, ref.W6-212)
Acetonitrile Optima® LC/MS Grade, (Fisher Chemica, refA955-212)
Methanol Optima® LC/MS Grade, (Fisher Chemical, ref. A456-212)
Pharmethyl 96° ethyl alcohol (Cristalco, ref 11371014)
Sodium acetate trihydrate (Sigma Aldrich ref S8625)
Acetic acid (Carl Roth ref HN55.1)
Benzoic acid, Reference 242381 (Sigma Aldrich ref242381)
Monopotassium phosphate (Riedel de Haen, ref. 04243)
Dibasic sodium phosphate dihydrate (Sigma Aldrich ref 30435)
1-Phenyl-1,2-Propanedione (PPD) (Aldrich ref 223034)

### 4.4. Preparation of the Reagent Solutions

### Phosphate Buffer pH 7.4

Dissolve 0.178 g monopotassium phosphate and 0.955 g dibasic sodium phosphate dihydrate in 100 ml purified water. The pH is adjusted if necessary.

### Phosphate Buffer pH 7.4—Methanol

A mixture is made with 50 ml phosphate buffer pH 7.4 and 50 ml methanol.

### Solution of 1-Phenyl-1,2-Propanedione (PPD)

A solution of exactly 0.375% (w/V) PPD is made in the mixture buffer phosphate pH 7.4—methanol (50/50).

### 4.5. Preparation of a Solution of the Product to be Tested

A solution of approximately 0.25% (w/V) of the product to be tested is made in the mixture buffer phosphate pH 7.4—methanol (50/50).

Product to be tested: Alagebrium chloride (MedChem Express, ref. HY-106024B), Sagerinic acid (prepared according to example 5.1.).

### 4.6. Benzoic acid Calibration Range

### 4.6.1. Standard Solutions

A stock solution of approximately 0.08% (w/V) benzoic acid is made in ethyl alcohol. This solution is diluted 1:10 (V/V) in purified water (D).

(D) is then diluted 1:5, 2:5, 3:5 and 4:5 (V/V) in purified water. (D1, D2, D3 and D4).

D1, D2, D3, D4 and D are passed over PVDF filters with a porosity of 0.45 μm.

### 4.6.2 Mobile Phases

A: Sodium acetate buffer: weigh 3.18 g of sodium acetate trihydrate, add 10 ml of acetic acid and make up to 500 ml with “Optima” water, stirring until completely solubilized.

B: Acetonitrile

### 4.6.3 Analytical Conditions

Column: Ascentis® Express C18 15 cm×3.0 mm 2.7 μm

Flow rate: 0.5 ml/min at 25 °C

Mode: gradient (see table 1)

Detection: ultraviolet (UV) at 229 nm

Injections: 10 μl for solutions D1, D2, D3, D4 and D.

TABLE 1

| Time (min) | Mobile phase A | Mobile phase B |
|---|---|---|
| 0 | 90 | 10 |
| 0-15 | 90 → 75 | 10 → 25 |
| 15-17 | 75 → 90 | 25 → 10 |
| 17-30 | Isocratic at 90 | Isocratic at 10 |

A linear calibration line was established: area of the benzoic acid peak, with a retention time of 9.17 min and observed at 229 nm, as a function of the concentration in % (w/V).

### 4.7. Action on PPD

### 4.7.1. Operating Mode

A series of hermetically sealed pillboxes are filled with:

| | Control | Test |
|---|---|---|
| Product solution to be tested (ml) | 0 | 1 |
| PPD solution (ml) | 1 | 1 |
| Buffer Phosphate-Methanol (50/50) (ml) | 1 | 0 |

Stir after each addition.

0.05 ml is taken from each pillbox to measure the benzoic acid content at TO. The pillboxes are resealed and placed in an oven at 37 °C for a minimum of 21 to a maximum of 24 hours.

The same volume will be taken for dosing on leaving the oven.

The time at 37 °C must be exactly the same for the control and all the tests, so they are prepared as they go along.

The HPLC dosing for the benzoic acid are also carried out on an ongoing basis.

### 5.4.7.2. HPLC Dosing of the Released Benzoic Acid

The 0.05 ml samples are diluted 1:5 (V/V) in purified water, i.e. by adding 0.2 ml.

They are passed through 0.45 μm pore size PVDF filters before injection.

### 5.4.7.2.1. Analytical Conditions

Column: Ascentis® Express C18 15 cm×3.0 mm 2.7 μm

Flow rate: 0.5 ml/min at 25 °C

Mode: gradient (see table 2)

Detection: ultraviolet (UV) at 229 nm

Injections: 10 μl

TABLE 2

| Time (min) | Mobile phase A | Mobile phase B |
|---|---|---|
| 0 | 90 | 10 |
| 0-12 | 90 → 78 | 10 → 22 |
| 12-14 | 78 → 0 | 22 → 100 |
| 14-20 | Isocratic at 0 | 100% isocratic |
| 20-22 | 0 → 90 | 100 → 10 |
| 22-35 | Isocratic at 90 | Isocratic at 10 |

### 4.8 Results

Using the calibration line, calculate the benzoic acid content in g/100 ml of reaction medium for
PPD control at TO and 24 hours: T_{T0} and T₂₄ₕ
test at TO and 24 hours: E_{T0} and E₂₄ₕ

These concentrations are converted into pmol benzoic acid/ml reaction medium (PM benzoic acid=122.12)
T_{T0}×10000/122.12

The concentration of PPD in pmol/ml of reaction medium is as follows:
0.375×1000000/100×2×148.16=12.655 (PM PPD=148.16)

The % of pmol of benzoic acid/μmol of PPD in the reaction medium is calculated:
T_{T0}×10000×100/122.12×12.655

The values obtained for the control and the trial are compared at different times.

The deglycating activity of sagerinic acid was compared with that of Alagebrium as a reference molecule.

| Molecule tested | Activity (arbitrary dimensionless unit) |
|---|---|
| Alagebrium (reference) | 28.79 |
| Sagerinic acid | 16.78 |

These results show that, in addition to its excellent activity in eliminating free radicals, saregenic acid has good protein deglycating activity.

### Example 5: Evaluation of the Effects of Sagerinic Acid and Resveratrol on Sirtuin-1

### 5.1. Principle

The SIRT1 Inhibitor/Activator screening kit deacetylates the substrate with SIRT1, followed by cleavage of the deacetylated substrate to release the fluorescent group, which is detected by fluorimetry at Ex/Em=400/505 nm.

The SIRT1 activator enhances the SIRT1 activity resulting in a higher fluorescent signal compared to the control. This kit provides a rapid, simple, sensitive and reliable test, suitable for high-throughput screening of SIRT1 activators.

### 5.2. Apparatus and Materials

Gemini EM spectrofluorometer
Stopwatch
Standard laboratory equipment.
Precision scales: ML104 (Mettler Toledo).
Automatic variable volume pipettes
Oven Innovens 28EU1 Jouan/Thermo

### 5.3 Reagents

Solutions of the kit: Reference ab283377 (Abeam)
Buffer
Substrate
SIRT1
NAD
DTT
DMSO Réference 34869 (Honeywell Riedel-de-Haen)

### 5.4. Preparation of the Reagent Solutions

SIRT1 enzyme: Store at −80 °C

Substrate: Store at −20 °C. Avoid repeated freezing/thawing. Use a fresh aliquot each time.

Mix 1 μl of substrate and 9 μl of DMSO.

NAD: Store at −80 °C. Avoid repeated freezing/thawing.

Mix 2 μl of NAD stock solution with 58 μl of DTT-free buffer.

1 M DTT: Store at −20 °C. Thaw and keep on ice during use.

Buffer: Store at 4 °C or −20 °C. Heat to 37 °C. Must be prepared just before use.

Preparation of the DTT buffer solution: mix 0.002 ml of DTT with 0.998 ml of buffer.

Revealing: Store at −20 °C. Avoid repeated freezing/thawing. Keep on ice during use.

### 5.5. Preparation of a Solution of the Product to be Tested (Activator)

A solution of exactly 2.28% (w/V) activator (resveratrol (Sigma Aldrich, ref.R5010) and sagerinic acid prepared according to example 5.1.) is made in DMSO. Dilute to 1:250 in the DTT buffer solution.

### 5.6. Operating Mode

Preparation of the enzyme solution: Make the following additions to 3 separate wells:

| | |
|---|---|
| SIRT1 dosing buffer (with DTT) | 23 μL |
| Enzyme SIRT1 | 2 μL |

### Screening Compounds, Inhibitor Control, Enzyme Control and Blank Control Preparations:

1. To the SIRT1 enzyme solution wells, add:
   25 μl of undiluted activator solution (2.28% solution) in a well as activator control (AC)
   25 μl of DTT-free buffer in a well as an enzyme control (EC)
   25 μl of diluted activator solution as assay (S)
2. In another well, add 50 μl of DTT-free buffer as a blank control (without enzyme).
3. Mix well and incubate the plaque for 5 minutes at 37 °C

Preparation of the substrate: After incubation, make the following additions to each well:

| Article | Volume |
|---|---|
| SIRT1 dosing buffer (with DTT) | 36 μL |
| Diluted substrate | 2 μL |
| Diluted NAD | 2 μL |

Mix and incubate at 37 °C for 30-60 min.

### Developing:

Add 10 μl of revealing to each well. Mix well and incubate for 10 min at 37 °C, protected from light.

### The Measure:

Read the fluorescence (Ex/Em=400/505 nm).

### 5.7. Results

The activation of sirtuin-1 by sagerinic acid was compared with that of resveratrol as a reference molecule.

| Molecule tested | Activity (arbitrary dimensionless unit) |
|---|---|
| Resveratrol (reference) | 80.54 |
| Sagerinic acid | 26.56 |

These results show that the sagerinic acid has a good effect on sirtuin activation, i.e. a good effect in preventing and/or treating the alteration in the DNA, as well as a good protein deglycating activity and excellent free radical eliminating activity.

### Example 6: Evaluation of the Effects of the Sagerinic Acid and Witch Hazel as Protectors of the Proteins of the Connective Tissue

### 6.1. Principle

A collagenase activity analysis kit (Sigma Aldrich MAK293) is used. The kit allows to screen collagenase inhibitors by measuring collagenase activity using a synthetic peptide (FALGPA) that mimics the structure of the collagen.

### 6.2. Apparatus and Materials

Jasco V-730 Dual Beam UV-Visible Spectrophotometer
Stopwatch
Standard laboratory equipment.
Precision scales: ML104 (Mettler Toledo).
Automatic variable volume pipettes
Oven Innovens 28EU1 Jouan/Thermo

### 6.3. Reagents

Solutions of the kit: Reference MAK293-1KT (Sigma Aldrich)
Collagenase buffer (MAK293A-KC)
Collagenase solution (MAK293B-KC)
Collagenase substrate (MAK293C-KC)
Pharmethyl 96° ethyl alcohol, Reference 11371014 (Cristalco)
Purified water

### 6.4. Preparation of a Solution of the Product to be Tested (Inhibitor)

A solution is made of exactly 1% (w/V) hamamelitannin (Extrasynthesis ref. 0958) in ethyl alcohol.

A solution of exactly 1% (w/V) sagerinic acid (prepared according to example 5.1.) is made in purified water.

### 6.5. Operating Mode

### 6.5.1. Control Solution

A series of hermetically sealed pillboxes are filled with:

| | Blank 1 | Control | Blank 2 | Test |
|---|---|---|---|---|
| Buffer (μl) | 320 | 300 | 316 | 296 |
| Collagenase (μl) | 0 | 20 | 0 | 20 |
| Substrate (μl) | 80 | 80 | 80 | 80 |
| Inhibitor (μl) | 0 | 0 | 4 | 4 |

After addition, stir quickly by hand
Place in an oven at 37 °C for 30 minutes
Add 1.6 mL of purified water. Mix well.
Read the DO: Sample against blank, spectrophotometer at 345 nm.

### 6.5.2. Results

### Activity on Collagenases

The anti-collagenase activity of sagerinic acid was compared with that of hamamelitannin as a reference molecule.

In absolute values: Y=[(DO_{Blank 2}−DO_{Test})−(DO_{Blank 1}−DO_{control})]/(DO_{Blank 1}−DO_{control})]×100

| | Activity (arbitrary |
|---|---|
| Molecule tested | dimensionless unit) |
| Hamamelitannin (reference) | 21.43 |
| Sagerinic acid | 46.43 |

These results show that the sagerinic acid has an inhibitory effect on proteases, represented here by collagenases, more than 2 times greater than that of the reference molecule, hamamelitannin.

This is in addition to excellent free radical eliminating activity, good activity on sirtuin activation, i.e. a good effect on preventing and/or treating the alteration in the DNA, and good protein deglycating activity.

Sagerinic acid therefore has an excellent activity on its own in preventing and/or treating disorders related to senescence in humans and animals, because it acts simultaneously on the four fundamental aspects of ageing oxidative stress, thanks to its excellent anti-radical action, protein glycation, thanks to its ability to reverse the glycation already present in proteins; DNA protection, thanks to its ability to activate sirtuins; and protection of connective tissue, thanks to its excellent inhibition of the proteases that degrade it, in particular elastases and collagenases.

The application of the sagerinic acid to the treatment of the four main axes of ageing will be based on doses defined in relation to what would be necessary to administer the four reference molecules used together.

The recommended daily oral dose for ascorbic acid is between 200 mg and 1 g per day (24), for resveratrol between 250 mg and 1 g per day (25), for hamamelitannin between 700 mg and 2 g per day (26 and 27) and for alagebrium between 100 mg and 300 mg per day (28).

A daily oral dose of sagerinic acid of between 10 mg and 2 g per day provides the recommended quantities of ascorbic acid, resveratol, hamamelitannin and alagebrium.

One advantage of the invention is that a single dose of sagerinic acid corresponds to separate doses of ascorbic acid, resveratrol, alagebrium and hammamelitannin.

### Example 7. Preparation of Melissa officinalis Extract Capsules Enriched with Sagerinic Acid

1 kg of Melissa officinalis dry leaf extract obtained according to Example 2 is intimately mixed with 10 g of sodium carbonate (E500) in a knife grinder.

This mixture is then used to fill No. 00 capsules (0.9 mL). Each capsule contains 550 mg of extract, equivalent to 44 mg of sagerinic acid.

### Example 8. Galenic Formula of a Gel Configured to Treat Aesthetic Disorders, Suitable for Use on the Face and on the Body

| | |
|---|---|
| Purified water: | 91.69% |
| Carbomer (Carbopol 980, Lubrizol): | 2% |
| Melissa officinalis dry leaf extract as described in example 1: | 5% |
| Benzyl alcohol (Geogard 221, Lonza): | 0.87% |
| Dehydroacetic acid (Geogard 221, Lonza): | 0.09% |
| Sodium hydroxide (Sigma-Aldrich): | 0.35% |

### BIBLIOGRAPHICAL REFERENCES

1. Wickens A P, Ageing and the free radical theory. Respir Physiol., 2001, 128(3), 379-91.
2. Grabowska W, Sikora E, Bielak-Zmijewska A. Sirtuins, a promising target in slowing down the ageing process. Biogerontology. 2017, 18(4), 447-476.
3. Ulrich P, Cerami A. Protein glycation, diabetes, and aging. Recent Prog Homr Res. 2001, 56, 1-21.
4. John E. Hodge Chemistry of Browning Reactions in Model Systems. Agricultural and food chemistry, 1953, 1 (15), 928-943.
5. Wautier J. L., Guillausseau P. J. Advanced Glycation end products, their receptors and diabetic angiopathy. Diabetes Metab (Paris), 2001, 27, 535-542.
6. Sakata N., Noma A., Yamamoto Y., Okamoto K., Meng J., Takebayashi S., Nagai R. and Horiuchi S. Modification of elastin by pentosidine is associated with the calcification of aortic redia in patients with cnd-stage renal discase. Nephrol. Dial. Transplant. 2003, 18, 1601-1609
7. Pageon H., Asselineau D. An in vitro approach to the chronological aging of skin by glycation of the collagen. Ann. N.Y. Acad. Sci. 2005, 1043, 529-532
8. Sell D. R. et al. Longitudinal determination of skin collagen glycation and glycoxydation rates predicts early death in C57BL/6NNIA mice. FASEB Journal, 2000, 14, 145-156
9. Zureik M, Robert L. Courbon D, Touboul P J, Bizbiz L, Ducinctiére P. Serum elastase activity, serum elastase inhibitors, and occurrence of carotid atherosclerotic plaques: the Etude sur le Vieillissement Artériel (EVA) study. Circulation. 2002, 105(22), 2638-45.
10. Monnier V. M. Intervention against the Maillard reaction in vivo. Archives of Biochemistry and Biophysics 419 (2003) 1-15
11. Jean D, Pouligon M. Utilisation de composés phénoliques pour la déglycation des protéines. WO2011042890A2, 2009.
12. Han Dai, David A. Sinclair, James L. Ellis, and Clemens Steegborn Sirtuin activators and inhibitors: Promises, achievements, and challenges. Pharmacol Ther. 2018, 188, 140-154.
13. Luigi Sartor, Elga Pezzato, Isabella Dell'Aica, Rosamaria Caniato, Susan Biggin, Spiridione Garbisa, Inhibition of matrix-proteases by polyphenols: chemical insights for anti-inflammatory and anti-invasion drug design, Biochemical Pharmacology, 2002, 64 (2), 229-237
14. Mohammed A. Dahab, Mostafa M. Hegazy, Hatem S. Abbass Hordatines as a Potential Inhibitor of COVID-19 Main Protease and RNA Polymerase: An In-Silico Approach. Natural Products and Bioprospecting, 2020, 10, 453-462.
15. Chi-Huey Wong, Yih-Shyun E. Cheng, Hui-Ming Yu, Ting-Jen R. Cheng, Chung-Yi Wu, Jim-Min Fang Compositions and methods for treating inflammation and inflammation-related disorders by plectranthus amboincus extracts. U.S. Pat. No. 8,105,636 B2.
16. Oliver A. Radtke, Lai Yeap Foo, Yinrong Lu, Albrecht F. Kiderlen, and Herbert Kolodziej Evaluation of Sage Phenolics for Their Antileishmanial Activity and Modulatory Effects on Interleukin-6, Interferon and Tumour Necrosis Factor-α-Release in RAW 264.7 Cells. Z. Naturforsch. 2003, 58c, 395-400.
17. Yinrong Lu, L. Yeap Foo Rosmarinic acid derivatives from Salvia officinalis. Phytochemistry, 1999, 51, 91-94.
18. Tzu-Ting Kuo, Hsin-Yi Chang, Tai-Yuan Chen, Bai-Chia Liu, Hsin-Yi Chen, Yuan-Chin Hsiung, Shih-Min Hsia, Chun-Ju Chang, and Tsui-Chin Huang Melissa officinalis Extract Induces Apoptosis and Inhibits Migration in Human Colorectal Cancer Cells. ACS Omega 2020, 5, 31792-31800.
19. Yashaswini Sharma, Ravikishore Velamuri, John Fagan and Jim Schaefer Full-Spectrum Analysis of Bioactive Compounds in Rosemary (Rosmarinus officinalis L.) as Influenced by Different Extraction Methods. Molecules 2020, 25, 4599.
20. Nitra Nuengchamnong, Kamrai Krittasilp, Kornkanok Ingkaninan Characterization of phenolic antioxidants in aqueous extract of Orthosiphon grandiflorus tea by LC-ESI-MS/MS coupled to DPPH assay. Food Chemistry, 2011, 127, 1287-1293.
21. Hong Ngoc Thuy Pham, Quan Van Vuong, Michael C. Bowyer, Christopher J. Scarlett In vitro anti-pancreatic cancer activity of HPLC-derived fractions from Helicteres hirsuta Lour. Stem. Mol Biol Rep, 2020, 47(2), 897-905.
22. Rhabar S. Breakers of advanced glycation Endproducts. U.S. Pat. No. 6,787,566B2, 1999.
23. Cerami A., Ulrich P. C., Wagle D. R., Hwang S., Vasan S., Egan J. J. Preventing and reversing the formation of advanced glycosylation Endproducts. U.S. Pat. No. 7,022,719 B2, 2006.
24. Levine M. Conry-Cantilena C, Wang Y, Welch R W, Washko P W, Dhariwal K R. Park J B, Lazarev A, Graumlich J F, King J, Cantilena L R. Vitamin C pharmacokinetics in healthy volunteers: evidence for a recommended dietary allowance. Proc Natl Acad Sci USA. 1996, 93(8), 3704-9.
25. https://www.drugs.com/npp/resveratrol.html
26. E/S/C/O/P Monographs. Online Series. Hamamelidis cortex. 2012
27. Vennat B, Pourrat H, Pouget M P, Gross D, Pourrat A. Tannins from Hamamelis virginiana: identification of proanthocyanidins and hamamelitannin quantification in leaf, bark, and stem extracts. Planta Med. 1988, 54(5), 454-7.
28. https://www.hopkinsmedicine.org/Press_releases/2005/11_15b_05.html

1. A sagerinic acid for use in the prevention and/or the treatment of the disorders related to senescence in a human or animal.

2. The sagerinic acid as claimed in claim 1, characterized in that the disorder is due to oxidative stress.

3. The sagerinic acid as claimed in claim 1, characterized in that the disorder is due to protein glycation.

4. The sagerinic acid according to claim 1, characterized in that the disorder is due to an alteration in the DNA.

5. The sagerinic acid as claimed in claim 1, characterized in that the disorder is due to an alteration in the proteins of the arterial connective tissue.

6. The sagerinic acid as claimed in claim 1, for use as an antioxidant agent and/or free radical trap.

7. The sagerinic acid as claimed in claim 1, for use as a protein deglycation agent.

8. The sagerinic acid as claimed in claim 1, for use as a DNA protection agent.

9. The sagerinic acid as claimed in claim 1, for use as a protease inhibition agent.

10. The sagerinic acid as claimed in claim 1, for use in the prevention and/or the treatment, simultaneously, of disorders due to oxidative stress, and disorders due to protein glycation, and disorders due to an alteration in the DNA and disorders due to an alteration in the proteins of the arterial connective tissue, in a human or animal.

11. A composition comprising sagerinic acid for use in the prevention and/or the treatment of disorders related to senescence in a human or animal.

12. The composition according to claim 11, characterized in that the disorder is due to oxidative stress.

13. The composition according to claim 11, characterized in that the disorder is due to protein glycation.

14. The composition according to claim 11, characterized in that the disorder is due to an alteration in the DNA.

15. The composition according to claim 11, characterized in that the disorder is due to an alteration in the proteins of the arterial connective tissue.

16. A composition comprising sagerinic acid for use in the simultaneous prevention and/or treatment of disorders due to an oxidative stress, and disorders due to protein glycation, and disorders due to an alteration in the DNA and disorders due to an alteration in the proteins of the arterial connective tissue, in a human or animal.
